(19) United States
(12) Patent Application Publication
(10) Pub. No.: US 2023/0080048 A1
(43) Pub. Date: Mar. 16, 2023

(54) METHOD AND APPARATUS FOR GENERATING A CONTAGION PREVENTION HEALTH ASSESSMENT

(71) Applicant: Specialty Diagnostic (SDI) Laboratories, Inc., Garden Grove, CA (US)

(72) Inventor: Ozman Mohiuddin, Redmond, WA (US)

(73) Assignee: Specialty Diagnostic (SDI) Laboratories, Inc., Garden Grove, CA (US)

(21) Appl. No.: 17/946,411

(22) Filed: Sep. 16, 2022

Related U.S. Application Data
(60) Provisional application No. 63/245,054, filed on Sep. 16, 2021.

(51) Int. Cl. G16H 50/20; G16H 50/80; G06F 21/32; G06V 40/20

(52) U.S. Cl. CPC G16H 50/20; G16H 50/80; G06F 21/32; G06V 40/20

(57) ABSTRACT

Aspects relate to generating a contagion prevention health assessment. An exemplary apparatus includes an optical device, at least a processor communicatively connected to the optical device and a memory communicatively connected to the at least a processor and to the optical device; the processor configured to receive an authentication datum from a user, authenticate the user as a function of the authentication datum and scan the user as a function of the authentication, where scanning the user further includes using a motion recognition machine learning model, generate a guidance datum as a function of the scan and to receive at least a user datum. and generate a health assessment as a function of a contagion status machine learning model, where the contagion status machine learning model is configured to receive the guidance datum and the at least a user datum as input and output the health assessment.

### FIELD OF THE INVENTION

The present invention generally relates to the field of disease contagion prevention assessment. In particular, the present invention is directed to self-testing procedures.

### BACKGROUND

The spread of infectious diseases, such as COVID-19, can be prevented by proper testing and precautions based on the test result.

### SUMMARY OF THE DISCLOSURE

In an aspect, an apparatus for generating a contagion prevention health assessment, the apparatus including an optical device, at least a processor communicatively connected to the optical device and a memory communicatively connected to the at least a processor and to the optical device, the memory containing instructions configuring the at least a processor to receive an authentication datum from a user. The memory also containing instructions further configuring the at least a processor to authenticate the user as a function of the authentication datum and scan the user as a function of the authentication, where scanning the user further includes using a motion recognition machine learning model. The memory also containing instructions further configuring the at least a processor to generate a guidance datum as a function of the scan, determine a scan status as a function of the guidance datum and to receive at least a user datum. The memory also containing instructions further configuring the at least a processor to generate a health assessment as a function of a contagion status machine learning model, where the contagion status machine learning model is configured to receive the guidance datum and the at least a user datum as input and output the health assessment.

In another aspect, a method for generating a contagion prevention health assessment, the method including receiving, by a processor, an authentication datum from a user and authenticating, by the processor, the user as a function of the authentication datum. The method also including scanning the user, by the processor communicatively connected to an optical device, as a function of the authentication, where scanning the user further comprises using a motion recognition machine learning model, and generating, by the processor, a guidance datum as a function of the scan. The method also including determining a scan status as function of the guidance datum and receiving, by the processor, at least a user datum, and generating, by the processor, a health assessment as a function of a contagion status machine learning model, where the contagion status machine learning model is configured to receive the guidance datum and the at least a user datum as input and output the health assessment.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for generating a contagion prevention health assessment;

FIG. 2 is an exemplary embodiment of a chatbot;

FIG. 3 is an exemplary embodiment of a scan the user;

FIG. 4 is an illustrative embodiment of a machine learning model;

FIG. 5 is an exemplary embodiment of a neural network;

FIG. 6 is an illustrative embodiment of a node of a neural network;

FIG. 7 is a graph illustrating exemplary fuzzy sets;

FIG. 8 is an exemplary flowchart of a method for generating a contagion prevention health assessment; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for generating a contagion prevention health assessment. In an embodiment, a contagion prevention health assessment is generated for the user based on user data and a guidance datum generated as a function of scanning the user. In an embodiment, the contagion prevention health assessment is generated using a contagion status machine learning model.

Aspects of the present disclosure can be used to ensure proper testing and prevent false negatives. Aspects of the present disclosure can also be used to authenticate a user based on user login data and a unique token generated by scanning a quick read (“QR”) code, which may be included with a test kit. This is so, at least in part, because the contagion status machine learning model takes data associate with the user in conjunction with a guidance datum generated based on scanning the user.

Aspects of the present disclosure allow for at home testing for diseases, such as COVID-19, that provides a level of precision that is closer to a level of precision provided by testing performed by a healthcare worker, or at a laboratory. Exemplary embodiments illustrating aspects of the present disclosure are described below in the context of several specific examples.

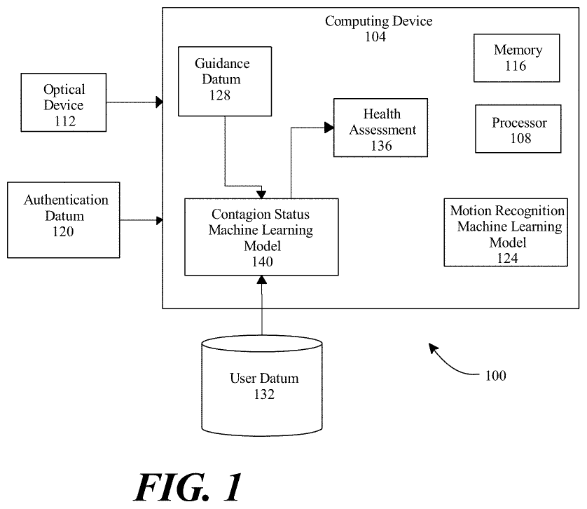

Referring now to FIG. 1, an exemplary embodiment of an apparatus 100 for generating a contagion prevention health assessment is illustrated. Apparatus 100 includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of apparatus 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

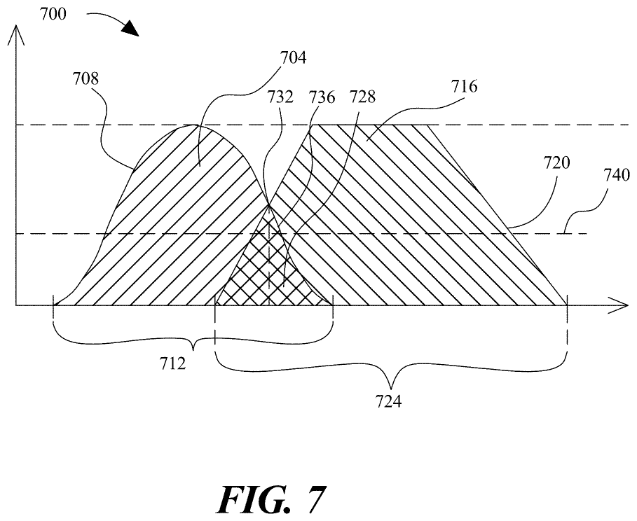

With continued reference to FIG. 1, apparatus 100 and/or computing device 104 includes at least a processor 108. The at least a processor 108 may be consistent with any processor discussed with reference to FIG. 7. Processor and at least a processor are used interchangeably throughout this disclosure. Processor 108 may include one processor. Processor 108 may include a plurality of processors. Apparatus 100 and/or computing device 104 includes an optical device 112 wherein the processor 108 is communicatively connected to optical device 112. In an embodiment, optical device 112 is used to determine user movement patterns. As used herein, “user movement patterns” may refer to movements performed by a user when performing a test for a disease. In an exemplary embodiment, user movement patterns may include hand movements, arm movements, head movements, object motion, such as a swab being held by the user, and the like.

With continued reference to FIG. 1, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Continuing to reference FIG. 1, optical device 112 may include any device capable of processing an image. In some embodiments, optical device 112 may include at least a camera. As used in this disclosure, a “camera” is a device that is configured to sense electromagnetic radiation, such as without limitation visible light, and generate an image representing the electromagnetic radiation. In some cases, a camera may include one or more optics. Exemplary non-limiting optics include spherical lenses, aspherical lenses, reflectors, polarizers, filters, windows, aperture stops, and the like. In some cases, at least a camera may include an image sensor. Exemplary non-limiting image sensors include digital image sensors, such as without limitation charge-coupled device (CCD) sensors and complimentary metal-oxide-semiconductor (CMOS) sensors, chemical image sensors, and analog image sensors, such as without limitation film. In some cases, a camera may be sensitive within a non-visible range of electromagnetic radiation, such as without limitation infrared. As used in this disclosure, “image data” is information representing at least a physical scene, space, and/or object. In some cases, image data may be generated by a camera. “Image data” may be used interchangeably through this disclosure with “image,” where image is used as a noun. An image may be optical, such as without limitation where at least an optic is used to generate an image of an object. An image may be material, such as without limitation when film is used to capture an image. An image may be digital, such as without limitation when represented as a bitmap. Alternatively, an image may be comprised of any media capable of representing a physical scene, space, and/or object. Alternatively, where “image” is used as a verb, in this disclosure, it refers to generation and/or formation of an image.

Still referring to FIG. 1, in some embodiments, apparatus 100 and/or computing device 104 communicatively connected to optical device 112 may include a machine vision system that includes at least a camera. A machine vision system may use images from at least a camera, to make a determination about a scene, space, and/or object. For example, in some cases a machine vision system may be used for world modeling or registration of objects within a space. In some cases, registration may include image processing, such as without limitation object recognition, feature detection, edge/corner detection, and the like. Non-limiting example of feature detection may include scale invariant feature transform (SIFT), Canny edge detection, Shi Tomasi corner detection, and the like. In some cases, registration may include one or more transformations to orient a camera frame (or an image or video stream) relative a three-dimensional coordinate system; exemplary transformations include without limitation homography transforms and affine transforms. In an embodiment, registration of first frame to a coordinate system may be verified and/or corrected using object identification and/or computer vision, as described above. For instance, and without limitation, an initial registration to two dimensions, represented for instance as registration to the x and y coordinates, may be performed using a two-dimensional projection of points in three dimensions onto a first frame, however. A third dimension of registration, representing depth and/or a z axis, may be detected by comparison of two frames; for instance, where first frame includes a pair of frames captured using a pair of cameras (e.g., stereoscopic camera also referred to in this disclosure as stereo-camera), image recognition and/or edge detection software may be used to detect a pair of stereoscopic views of images of an object; two stereoscopic views may be compared to derive z-axis values of points on object permitting, for instance, derivation of further z-axis points within and/or around the object using interpolation. This may be repeated with multiple objects in field of view, including without limitation environmental features of interest identified by object classifier and/or indicated by an operator. In an embodiment, x and y axes may be chosen to span a plane common to two cameras used for stereoscopic image capturing and/or an xy plane of a first frame; a result, x and y translational components and ϕ may be pre-populated in translational and rotational matrices, for affine transformation of coordinates of object, also as described above. Initial x and y coordinates and/or guesses at transformational matrices may alternatively or additionally be performed between first frame and second frame, as described above. For each point of a plurality of points on object and/or edge and/or edges of object as described above, x and y coordinates of a first stereoscopic frame may be populated, with an initial estimate of z coordinates based, for instance, on assumptions about object, such as an assumption that ground is substantially parallel to an xy plane as selected above. Z coordinates, and/or x, y, and z coordinates, registered using image capturing and/or object identification processes as described above may then be compared to coordinates predicted using initial guess at transformation matrices; an error function may be computed using by comparing the two sets of points, and new x, y, and/or z coordinates, may be iteratively estimated and compared until the error function drops below a threshold level. In some cases, a machine vision system may use a classifier, such as any classifier described throughout this disclosure. An exemplary machine vision camera is an OpenMV Cam H7 from OpenMV, LLC of Atlanta, Ga., U.S.A. OpenMV Cam comprises a small, low power, microcontroller which allows execution of machine vision applications. OpenMV Cam comprises an ARM Cortex M7 processor and a 640×480 image sensor operating at a frame rate up to 150 fps. OpenMV Cam may be programmed with Python using a Remote Python/Procedure Call (RPC) library. OpenMV CAM may be used to operate image classification and segmentation models, such as without limitation by way of TensorFlow Lite; detection motion, for example by way of frame differencing algorithms; marker detection, for example blob detection; object detection, for example face detection; eye tracking; person detection, for example by way of a trained machine learning model; camera motion detection, for example by way of optical flow detection; code (barcode) detection and decoding; image capture; and video recording.

With continued reference to FIG. 1, apparatus 100 and/or computing device 104 includes a memory 116 communicatively connected to the at least a processor 108, wherein the memory 116 contains instructions configuring the processor 108 to perform tasks in accordance with this disclosure. In an embodiment, memory 116 contains instructions configuring processor 108 to receive an image datum from optical device 112. “Image datum,” as used herein, is data, or an element of data, that constitutes a representation of an image received from optical device 112. In an embodiment, image datum may include a recording of the user. In another embodiment, image datum may be a live-feed image transmitted by optical device.

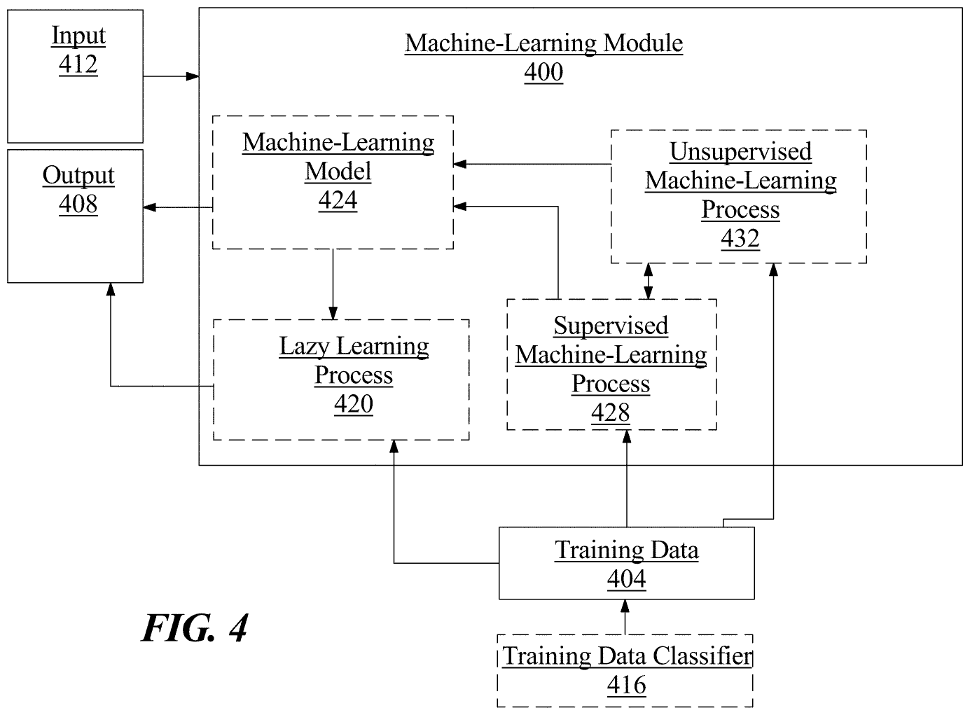

Continuing to refer to FIG. 1, memory 116 contains instructions configuring processor 108 to receive an authentication datum 120 from a user. As used in this disclosure, an “authentication datum” is an element of data that uniquely identifies a user. In an embodiment, authentication datum may be encrypted. In some embodiments, processor 108 may be further configured to scan a QR code using optical device 112. In an embodiment, processor 108 may be further configured to generate an authentication token as a function of the QR code scan. In an embodiment, authentication datum may include an authentication token. In an embodiment, receiving the authentication datum 120 may include scanning the user's face. A nonlimiting example of an authentication that includes a facial scan is the Face ID feature, made by Apple Inc., headquartered in Cupertino, Calif. USA. In an embodiment, authentication datum 120 that include scanning the user's face may be generated using an image classifier. In another embodiment, authentication datum 120 through a facial scan may be generated using a neural network. In one embodiment, authentication datum 120 through facial scan may be generated by using a machine learning model. Image classification may include training a classifier, neural network and/or machine-learning model using sample image data. In an embodiment, sample image data may include a plurality of images related to the user. In an embodiment, sample image data may include images related to a plurality of users, where the sample image data of plurality users is used to train image classifier, neural network and/or machine-learning model to differentiate other images from images related to the user. Sample image data may include images labeled by the user. Sample image data may include images of the user previously recorded by optical device 112. Training may be performed consistent with any embodiment described in FIGS. 4-7.

Still referring to FIG. 1, memory 116 may contain instructions configuring processor 108 to generate the authentication datum as a function of a speech recognition machine learning model. Speech recognition machine learning model may be trained using speech pattern training data. Exemplary machine learning processes are described in detail with reference to FIGS. 4-7. As used in this disclosure, “speech pattern training data” is a training set that correlates speech parameters to speech patterns. In some cases, speech pattern training data may be compiled from historic information, for instance by a user. In some cases, speech pattern training data may be compiled by an unsupervised machine learning process. Speech pattern training data may use speech parameters correlated to speech patterns for one individual user, or for a cohort or population of users. Historic information may include information from speech-related study. In some cases, historical information may include information captured from use of apparatus 100 and/or computing device 104. Processor 108 may input speech pattern training data into a speech pattern machine learning algorithm. As used in this disclosure, a “speech recognition machine learning algorithm” is any machine learning algorithm that is configured to train a speech recognition machine learning model using speech pattern training data. Processor 108 may train a speech recognition machine learning model 140, as a function of speech pattern machine learning algorithm. As used in this disclosure, “speech recognition machine learning model” is a machine learning model that is configured to take as input at least a speech parameter, such as a user's spoken words, and output at least a correlated speech pattern. Processor 108 may determine at least a speech pattern unique to a user as a function of speech recognition machine learning model and at least a speech parameter for the user.

With continued reference to FIG. 1. As used in this disclosure, a “speech pattern” is a representation of a speech-related behavioral phenomenon. In some cases, a speech pattern may be derived or otherwise determined from a speech parameter. Exemplary speech patterns include timber, pitch, and cadence of speech. In some cases, speech pattern may be unrelated to content of a user's speech. Instead, in some cases, speech pattern may be related to changes audible characteristics of user's speech. In some cases, speech pattern may be derived through analysis of speech parameters, for instance audio analysis described above. Speech pattern may include one or more prosodic variables. As used in this disclosure, “prosodic variables” are variables that relate to spoken syllables or larger speech units. In some cases, speech pattern may include audible variables, for instance pitch, change in pitch, length of units of speech (e.g., syllables), volume, loudness, prominence (i.e., relative volume of a unit speech, timbre, quality of sound, and the like. In some cases, speech pattern may include acoustic terms. Acoustic terms may include without limitation fundamental frequency, duration, intensity, sound pressure, spectral characteristics, and the like. Speech pattern may include speech tempo. As used in this disclosure, “speech tempo” is a measure of a number of speech units within a certain amount of time. Speech tempo may vary within speech of one person, for instance according to context and emotional factors. Speech tempo may have units of syllables per second.

With continued reference to FIG. 1, memory 116 contains instructions configuring processor 108 to authenticate the user as a function of the authentication datum 120. In an embodiment, processor 108 may be further configured to authenticate the user as a function of the authentication token. Processor 108 may be configured to authenticate the user according with any of the embodiments described herein. Authentication of the user may include comparing user login information to user login input. Authentication of the user may further include comparing user features such as facial features, speech patterns, user movement patterns, and the like to an image datum of the user received from optical device 112. “Authentication token”, as used herein, is an unique identifier, or data element, that provides secure identification of a user, a product key, or a combination of both. As a nonlimiting example, processor 108 may generate an authentication token for the user as a function of the login credentials. In another nonlimiting example, processor 108 may scan a QR code in a test kit and generate an authentication token that is used to identify the test kit. In another example, without limitations, processor 108 may scan the QR code in a test kit and generate an authentication token as a function of the QR code and the user login credentials. Authentication and encryption/decryption methods are discussed in more detail further below.

In an embodiment, and still referring to FIG. 1. apparatus 100 and/or computing device 104 may perform or implement one or more aspects of a cryptographic system. In one embodiment, a cryptographic system is a system that converts data from a first form, known as “plaintext,” which is intelligible when viewed in its intended format, into a second form, known as “ciphertext,” which is not intelligible when viewed in the same way. Ciphertext may be unintelligible in any format unless first converted back to plaintext. In one embodiment, a process of converting plaintext into ciphertext is known as “encryption.” Encryption process may involve the use of a datum, known as an “encryption key,” to alter plaintext. Cryptographic system may also convert ciphertext back into plaintext, which is a process known as “decryption.” Decryption process may involve the use of a datum, known as a “decryption key,” to return the ciphertext to its original plaintext form. In embodiments of cryptographic systems that are “symmetric,” decryption key is essentially the same as encryption key: possession of either key makes it possible to deduce the other key quickly without further secret knowledge. Encryption and decryption keys in symmetric cryptographic systems may be kept secret and shared only with persons or entities that the user of the cryptographic system wishes to be able to decrypt the ciphertext. One example of a symmetric cryptographic system is the Advanced Encryption Standard (“AES”), which arranges plaintext into matrices and then modifies the matrices through repeated permutations and arithmetic operations with an encryption key.

Continuing to refer to FIG. 1. In embodiments of cryptographic systems that are “asymmetric,” either encryption or decryption key cannot be readily deduced without additional secret knowledge, even given the possession of a corresponding decryption or encryption key, respectively; a common example is a “public key cryptographic system,” in which possession of the encryption key does not make it practically feasible to deduce the decryption key, so that the encryption key may safely be made available to the public. An example of a public key cryptographic system is RSA, in which an encryption key involves the use of numbers that are products of very large prime numbers, but a decryption key involves the use of those very large prime numbers, such that deducing the decryption key from the encryption key requires the practically infeasible task of computing the prime factors of a number which is the product of two very large prime numbers. Another example is elliptic curve cryptography, which relies on the fact that given two points P and Q on an elliptic curve over a finite field, and a definition for addition where A+B=−R, the point where a line connecting point A and point B intersects the elliptic curve, where “0,” the identity, is a point at infinity in a projective plane containing the elliptic curve, finding a number k such that adding P to itself k times results in Q is computationally impractical, given correctly selected elliptic curve, finite field, and P and Q.

In some embodiments, and still referring to FIG. 1. apparatus 100 and/or computing device 104 may produce cryptographic hashes, also referred to by the equivalent shorthand term “hashes.” A cryptographic hash, as used herein, is a mathematical representation of a lot of data, such as files or blocks in a block chain as described in further detail below; the mathematical representation is produced by a lossy “one-way” algorithm known as a “hashing algorithm.” Hashing algorithm may be a repeatable process; that is, identical lots of data may produce identical hashes each time they are subjected to a particular hashing algorithm. Because hashing algorithm is a one-way function, it may be impossible to reconstruct a lot of data from a hash produced from the lot of data using the hashing algorithm. In the case of some hashing algorithms, reconstructing the full lot of data from the corresponding hash using a partial set of data from the full lot of data may be possible only by repeatedly guessing at the remaining data and repeating the hashing algorithm; it is thus computationally difficult if not infeasible for a single computer to produce the lot of data, as the statistical likelihood of correctly guessing the missing data may be extremely low. However, the statistical likelihood of a computer of a set of computers simultaneously attempting to guess the missing data within a useful timeframe may be higher, permitting mining protocols as described in further detail below.

With continued reference to FIG. 1. In an embodiment, hashing algorithm may demonstrate an “avalanche effect,” whereby even extremely small changes to lot of data produce drastically different hashes. This may thwart attempts to avoid the computational work necessary to recreate a hash by simply inserting a fraudulent datum in data lot, enabling the use of hashing algorithms for “tamper-proofing” data such as data contained in an immutable ledger as described in further detail below. This avalanche or “cascade” effect may be evinced by various hashing processes; persons skilled in the art, upon reading the entirety of this disclosure, will be aware of various suitable hashing algorithms for purposes described herein. Verification of a hash corresponding to a lot of data may be performed by running the lot of data through a hashing algorithm used to produce the hash. Such verification may be computationally expensive, albeit feasible, potentially adding up to significant processing delays where repeated hashing, or hashing of large quantities of data, is required, for instance as described in further detail below. Examples of hashing programs include, without limitation, SHA256, a NIST standard; further current and past hashing algorithms include Winternitz hashing algorithms, various generations of Secure Hash Algorithm (including “SHA-1,” “SHA-2,” and “SHA-3”), “Message Digest” family hashes such as “MD4,” “MD5,” “MD6,” and “RIPEMD,” Keccak, “BLAKE” hashes and progeny (e.g., “BLAKE2,” “BLAKE-256,” “BLAKE-512,” and the like), Message Authentication Code (“MAC”)-family hash functions such as PMAC, OMAC, VMAC, HMAC, and UMAC, Poly1305-AES, Elliptic Curve Only Hash (“ECOH”) and similar hash functions, Fast-Syndrome-based (FSB) hash functions, GOST hash functions, the Grøstl hash function, the HAS-160 hash function, the JH hash function, the RadioGatún hash function, the Skein hash function, the Streebog hash function, the SWIFFT hash function, the Tiger hash function, the Whirlpool hash function, or any hash function that satisfies, at the time of implementation, the requirements that a cryptographic hash be deterministic, infeasible to reverse-hash, infeasible to find collisions, and have the property that small changes to an original message to be hashed will change the resulting hash so extensively that the original hash and the new hash appear uncorrelated to each other. A degree of security of a hash function in practice may depend both on the hash function itself and on characteristics of the message and/or digest used in the hash function. For example, where a message is random, for a hash function that fulfills collision-resistance requirements, a brute-force or “birthday attack” may to detect collision may be on the order of O(2^{n/2}) for n output bits; thus, it may take on the order of 2²⁵⁶ operations to locate a collision in a 512 bit output “Dictionary” attacks on hashes likely to have been generated from a non-random original text can have a lower computational complexity, because the space of entries they are guessing is far smaller than the space containing all random permutations of bits. However, the space of possible messages may be augmented by increasing the length or potential length of a possible message, or by implementing a protocol whereby one or more randomly selected strings or sets of data are added to the message, rendering a dictionary attack significantly less effective.

Continuing to refer to FIG. 1, a “secure proof,” as used in this disclosure, is a protocol whereby an output is generated that demonstrates possession of a secret, such as device-specific secret, without demonstrating the entirety of the device-specific secret; in other words, a secure proof by itself, is insufficient to reconstruct the entire device-specific secret, enabling the production of at least another secure proof using at least a device-specific secret. A secure proof may be referred to as a “proof of possession” or “proof of knowledge” of a secret. Where at least a device-specific secret is a plurality of secrets, such as a plurality of challenge-response pairs, a secure proof may include an output that reveals the entirety of one of the plurality of secrets, but not all of the plurality of secrets; for instance, secure proof may be a response contained in one challenge-response pair. In an embodiment, proof may not be secure; in other words, proof may include a one-time revelation of at least a device-specific secret, for instance as used in a single challenge-response exchange.

Continuing to refer to FIG. 1. Secure proof may include a zero-knowledge proof, which may provide an output demonstrating possession of a secret while revealing none of the secret to a recipient of the output; zero-knowledge proof may be information-theoretically secure, meaning that an entity with infinite computing power would be unable to determine secret from output. Alternatively, zero-knowledge proof may be computationally secure, meaning that determination of secret from output is computationally infeasible, for instance to the same extent that determination of a private key from a public key in a public key cryptographic system is computationally infeasible. Zero-knowledge proof algorithms may generally include a set of two algorithms, a prover algorithm, or “P,” which is used to prove computational integrity and/or possession of a secret, and a verifier algorithm, or “V” whereby a party may check the validity of P. Zero-knowledge proof may include an interactive zero-knowledge proof, wherein a party verifying the proof must directly interact with the proving party; for instance, the verifying and proving parties may be required to be online, or connected to the same network as each other, at the same time. Interactive zero-knowledge proof may include a “proof of knowledge” proof, such as a Schnorr algorithm for proof on knowledge of a discrete logarithm. in a Schnorr algorithm, a prover commits to a randomness r, generates a message based on r, and generates a message adding r to a challenge c multiplied by a discrete logarithm that the prover is able to calculate; verification is performed by the verifier who produced c by exponentiation, thus checking the validity of the discrete logarithm. Interactive zero-knowledge proofs may alternatively or additionally include sigma protocols. Persons skilled in the art, upon reviewing the entirety of this disclosure, will be aware of various alternative interactive zero-knowledge proofs that may be implemented consistently with this disclosure.

Alternatively, and still referring to FIG. 1, zero-knowledge proof may include a non-interactive zero-knowledge, proof, or a proof wherein neither party to the proof interacts with the other party to the proof; for instance, each of a party receiving the proof and a party providing the proof may receive a reference datum which the party providing the proof may modify or otherwise use to perform the proof. As a non-limiting example, zero-knowledge proof may include a succinct non-interactive arguments of knowledge (ZK-SNARKS) proof, wherein a “trusted setup” process creates proof and verification keys using secret (and subsequently discarded) information encoded using a public key cryptographic system, a prover runs a proving algorithm using the proving key and secret information available to the prover, and a verifier checks the proof using the verification key; public key cryptographic system may include RSA, elliptic curve cryptography, ElGamal, or any other suitable public key cryptographic system. Generation of trusted setup may be performed using a secure multiparty computation so that no one party has control of the totality of the secret information used in the trusted setup; as a result, if any one party generating the trusted setup is trustworthy, the secret information may be unrecoverable by malicious parties. As another non-limiting example, non-interactive zero-knowledge proof may include a Succinct Transparent Arguments of Knowledge (ZK-STARKS) zero-knowledge proof. In an embodiment, a ZK-STARKS proof includes a Merkle root of a Merkle tree representing evaluation of a secret computation at some number of points, which may be 1 billion points, plus Merkle branches representing evaluations at a set of randomly selected points of the number of points; verification may include determining that Merkle branches provided match the Merkle root, and that point verifications at those branches represent valid values, where validity is shown by demonstrating that all values belong to the same polynomial created by transforming the secret computation. In an embodiment, ZK-STARKS does not require a trusted setup. Zero-knowledge proof may include any other suitable zero-knowledge proof. Zero-knowledge proof may include, without limitation bulletproofs. Zero-knowledge proof may include a homomorphic public-key cryptography (hPKC)-based proof. Zero-knowledge proof may include a discrete logarithmic problem (DLP) proof. Zero-knowledge proof may include a secure multi-party computation (MPC) proof. Zero-knowledge proof may include, without limitation, an incrementally verifiable computation (IVC). Zero-knowledge proof may include an interactive oracle proof (TOP). Zero-knowledge proof may include a proof based on the probabilistically checkable proof (PCP) theorem, including a linear PCP (LPCP) proof. Persons skilled in the art, upon reviewing the entirety of this disclosure, will be aware of various forms of zero-knowledge proofs that may be used, singly or in combination, consistently with this disclosure.

Still referring to FIG. 1. In an embodiment, secure proof is implemented using a challenge-response protocol. In an embodiment, this may function as a one-time pad implementation; for instance, a manufacturer or other trusted party may record a series of outputs (“responses”) produced by a device possessing secret information, given a series of corresponding inputs (“challenges”), and store them securely. In an embodiment, a challenge-response protocol may be combined with key generation. A single key may be used in one or more digital signatures as described in further detail below, such as signatures used to receive and/or transfer possession of crypto-currency assets; the key may be discarded for future use after a set period of time. In an embodiment, varied inputs include variations in local physical parameters, such as fluctuations in local electromagnetic fields, radiation, temperature, and the like, such that an almost limitless variety of private keys may be so generated. Secure proof may include encryption of a challenge to produce the response, indicating possession of a secret key. Encryption may be performed using a private key of a public key cryptographic system, or using a private key of a symmetric cryptographic system; for instance, trusted party may verify response by decrypting an encryption of challenge or of another datum using either a symmetric or public-key cryptographic system, verifying that a stored key matches the key used for encryption as a function of at least a device-specific secret. Keys may be generated by random variation in selection of prime numbers, for instance for the purposes of a cryptographic system such as RSA that relies prime factoring difficulty. Keys may be generated by randomized selection of parameters for a seed in a cryptographic system, such as elliptic curve cryptography, which is generated from a seed. Keys may be used to generate exponents for a cryptographic system such as Diffie-Helman or ElGamal that are based on the discrete logarithm problem.

Continuing to refer to FIG. 1, memory 116 contains instructions configuring processor 108, communicatively connected to optical device 112, to scan the user as a function of the authentication. In an embodiment, scanning the user further includes using a motion recognition machine learning model 124. Exemplary machine learning processes are described in detail with reference to FIGS. 4-7. In some cases, processor 104 may receive motion recognition training data. As used in this disclosure, “motion recognition training data” is a training set that correlates user movement patterns, such as, without limitation, arm movement patterns, hand movement patterns, head movement patterns, object movement patterns, and the like, to guidance motion patterns. As used in this disclosure, “guidance motion pattern” is a set of motions required to be done by the user and objects, such as a test swab, that ensures a sample of human specimen is properly collected for testing for a particular disease, such as COVID-19. As a nonlimiting example, guidance motion pattern includes an angle that the swab may be inserted into the left or right nostril, the depth that that it must be inserted and a number of rotations that the user must make with the swab while inserted in one of the nostrils. In an embodiment, guidance motion patterns may be captured using optical device 112, or similar devices. In an embodiment, motion recognition training data may include guidance motion patterns labeled by a user or a plurality of users. In some cases, motion recognition training data may be compiled and/or correlated from historic information, for instance by a user. In some instances, motion recognition training data may be compiled and or correlated by an unsupervised machine learning process. Motion recognition training data may use user movement patterns correlated to guidance motion patterns for one individual user, or for a cohort or population of users. In an embodiment, correlated motion patterns may include guidance motion patterns labeled by a user or a plurality of users. Historic information may include information from motion pattern-related study. In some cases, historical information may include information captured from use of apparatus 100. Processor 104 may input movement pattern training data into a motion recognition machine learning algorithm. As used in this disclosure, “motion recognition machine learning model” is a machine learning model that is configured to take as input at least a movement pattern, such as, without limitation, arm movement patterns, hand movement patterns, head movement patterns, object movement patterns and output at least a correlated guidance motion pattern. Processor 104, communicatively coupled to optical device 112, may determine a motion pattern as a function of motion recognition machine learning model 124 and one or more user movements. Motion recognition machine learning model may include any machine learning model described throughout this disclosure.

Still referring to FIG. 1, in some instances motion recognition machine learning model 124 may include a classifier, such as any classifier described in detail below. In some cases, classifier may include calculation of distance. For instance, classifier may employ fuzzy sets having coverages based on distances from different centroids/neighbors (e.g., probability of set membership based on degree of closeness to a corresponding classification). In some cases, classifier may include a fuzzy inference engine configured to determine one or more classification set membership probability determinations. Inference engine may include a sum-product, max-product, min-max engine, or the like. In some cases, classifier may perform one or more rules and/or actions as a function of output from fuzzy inference engine, thereby dealing with different overlapping fuzzy sets (i.e., classifications). Fuzzy set classification is described in greater detail with reference to FIG. 7 below.

With continued reference to FIG. 1, in some embodiments, processor 104 may be additionally configured to determine a confidence metric associated with correlation and/or determination of motion pattern. As used in this disclosure, a “confidence metric” is a quantified expression of confidence associated with a function, such as a likelihood or probability that an output of a function is accurate or correct. Determination of a confidence metric may include any appropriate process described in this disclosure, including for example with reference to FIGS. 4-7. Exemplary processes for determining a confidence metric include, without limitation, fuzzy mathematics (see FIG. 7). In some cases, a confidence metric may be a proportional or unitless figure, for example expressed in terms of a proportion or percentage. Alternatively of additionally, a confidence metric may be represented using relative or absolute units. In some cases, a confidence metric may be compared to a threshold confidence metric in order to determine suitability of an associated correlation and/or determination, for example of a motion pattern. For instance, in some cases a confidence metric no less than a threshold confidence metric of 95%, 90%, 85%, 75%, or 50% is required in order to assure an underlying correlation and/or determination of cognitive status is “correct.”

Continuing to refer to FIG. 1, memory 116 contains instructions configuring processor 108 to generate a guidance datum 128 as function of the scan. As used herein, “guidance datum” is a datum describing a correlation between user movement patterns and guidance motion patterns, as described above. In a nonlimiting example, guidance datum may include a binary status, such as “pass” for instances where the user movement pattern matches the guidance motion patterns. In a nonlimiting embodiment, apparatus 100 may be communicatively connected to a guidance database. Guidance database may include any database, or data structure, described in this disclosure. In another nonlimiting example, guidance datum 128 may include a confidence metric, as described above, that is compared to a set confidence threshold. In an embodiment, guidance datum 128 may be a degree of match using a distance metric classifier. In another embodiment, guidance datum 128 may be a degree of match using fuzzy matching. Fuzzy matching is described in detail in FIG. 7.

Still referring to FIG. 1, memory 116 contains instructions configuring processor 108 to determine a scan status as a function of the guidance datum 128. As used in this disclosure, a “scan status” is data or an element of data that signals whether the scan was performed correctly. In a nonlimiting example, scan status may be a “successful” status based on a confidence level threshold set by the guidance datum 128. In another nonlimiting example, scan status may be a “failed” based on the confidence level for the guidance datum 128 being below a set threshold.

Still referring to FIG. 1, memory 116 contains instructions configuring processor 108 to receive at least a user datum 132. As used herein, “user datum” includes a plurality of data associated with the user. In nonlimiting embodiments, user datum may include user's vaccination status, household type, number of individuals living with the user, existing medical conditions, possible exposures by user to the tested disease, prior testing results, and the like. In an embodiment, user datum may be stored in a database. In an embodiment, user datum 132 may be stored in an immutable sequential listing. In some embodiments, user datum 132 may include at least a test result datum. “Test result datum” may include any data, or element of data, describing the status of a test. In a nonlimiting example, test result datum may be an input by the user describing a positive or negative result. In a nonlimiting example, test result datum may be a checkbox where the user may select it if the test is positive. In another nonlimiting example, test result may be accessed through a database.

Still referring to FIG.1. In some embodiments, at least a user datum 132 may be received through a user input. “User input” as used in this disclosure is information pertaining to a user's actions. User input may include, but is not limited to, typing on a touch screen, voice inputs, clicking on icons, and the like. User input 124 may be received from a remote computing device. A “remote computing device” as used in this disclosure is a computing device external to a first computing device. A remote computing device may include, but is not limited to, smartphones, laptops, desktops, tablets, and the like. Computing device 104 may generate a digital access link, such as but not limited to, a hyperlink. A digital access link may allow a user to provide user input to computing device 104 from a remote computing device. In some embodiments, computing device 104 may be configured to receive user input over a server. In some embodiments, computing device 104 may receive user input through a mobile application. A mobile application may include software configured to run on a smartphone, tablet, laptop, and the like. A mobile application may generate a graphical user interface configured to receive user input such as, but not limited to, text boxes, icons, scrolling menus, and the like. Computing device 104 may be configured to interact with a user through a mobile application, such as, but not limited to, prompting a user to input user datum 132, providing test result data, and the like. In some embodiments, user input may be received directly on computing device 104.

Continuing to refer to FIG. 1, memory 116 contains instructions configuring processor 108 to generate health assessment 136 as a function of a contagion status machine learning model 140. “Health assessment,” as used in this disclosure, refers to data, or elements of data, describing the status of the user related to the disease being tested for. In a nonlimiting example, health assessment may include a report describing the possibility that the user received a false-negative test result. In a further nonlimiting example, a health assessment describing a high possibility of a false-negative test result may prompt the user to take a new test. In one embodiment, the contagion status machine learning model 140 is configured to receive the guidance datum 128 and the at least a user datum 132 as input and output the health assessment 136. As used herein, contagion status machine learning model is a machine learning model, consistent with any machine learning model described in this disclosure, configured to determine a contagion status for the user. “Contagion status”, as is used herein, includes any data, or element of data, describing the probability that the user contracted the disease that is being tested. Contagion status machine learning model may include any machine learning model, or algorithm, described throughout this disclosure. Exemplary machine learning processes are described in detail with reference to FIGS. 4-7.

Still referring to FIG. 1. In an embodiment, contagion status machine learning model 140 may be an infectious status machine learning model. For instance, and without limitation, contagion status machine learning model 140 and health assessment 136 may be consistent with the infectious status machine learning model and infection status of a user in U.S. patent application Ser. No. 17/566,869 and titled “SYSTEM AND METHOD FOR INFORMING A USER OF A COVID-19 INFECTION STATUS” which is incorporated herein in its entirety.

Still referring to FIG. 1. In some embodiments, memory 116 may contain instructions configuring processor 108 to generate health assessment 136 using a chatbot system. In a nonlimiting example, the chatbot system may prompt the user for a user input. In another nonlimiting example, chatbot may prompt user for user input that include the user datum. In another example, without limitations, chatbot may prompt user for an authentication datum. As used in this disclosure, “chatbot system” is a computer program designed to simulate conversation with a user. A chatbot may accomplish this by presenting the user with questions and/or prompts, such as prompting the user to perform a new scan. In an embodiment, a chatbot is designed and configured to simulate how a human would behave/respond in a conversation. In an embodiment, chatbot may generate responses as a function a machine learning model. Chatbot machine learning model may be implemented consistent with any embodiment described in this disclosure. The chatbot system may include any chatbot system described herein. In a nonlimiting example, chatbot may gather user datum by prompting the user for input. Chatbot system is described in further detail below.

Still referring to FIG. 1. In some embodiments, memory 116 may contain instructions further configuring processor 108 to prompt the user, using the chatbot system, for a second scan as a function of the guidance datum, perform the second scan of the user as a function of the prompt, wherein scanning the user further comprises using motion recognition machine learning model 124, and generate a second guidance datum as a function of the second scan. In an embodiment, memory 116 may contain instructions further configuring processor 108 to schedule a test appointment for the user as a function of the health assessment 136. Scheduling the test appointment may include accessing test center information on a database. Scheduling the test appointment may include generating a reminder task in a mobile device. In a nonlimiting example, processor 108 may be configured to access a database that includes information related to test centers and display the information to the user. In another nonlimiting example, processor 108 may further create a task reminder with the test center's information in the user's mobile device. Database is described in more detail below.

Still referring to FIG. 1, a database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

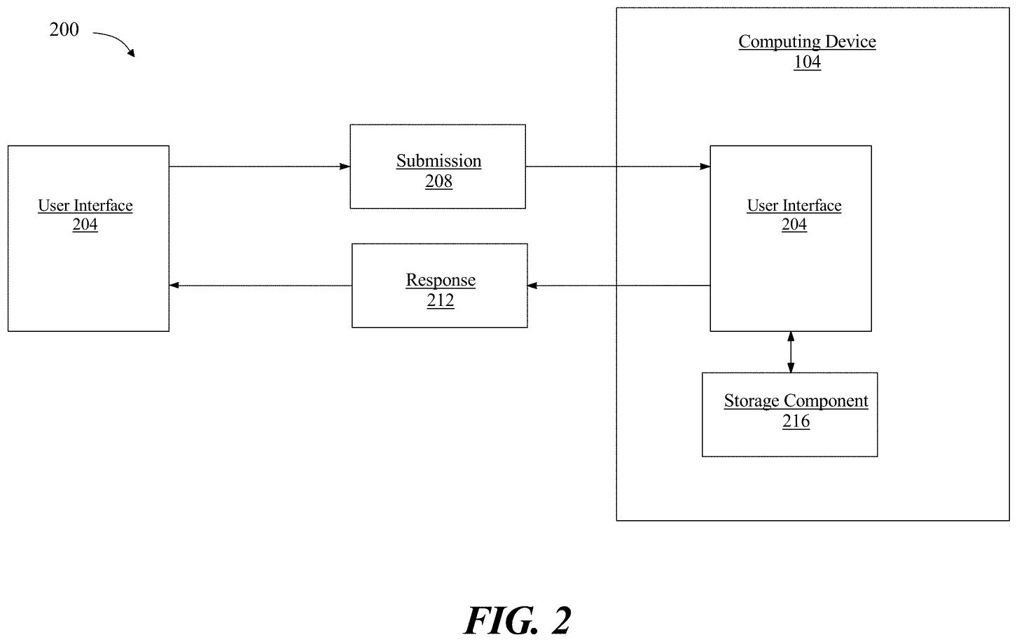

Referring to FIG. 2, a chatbot system 200 is schematically illustrated. According to some embodiments, a user interface 204 may be communicative with the computing device 104 configured to operate a chatbot. In some cases, user interface 204 may be local to computing device 104. Alternatively, or additionally, in some cases, user interface 204 may remote to computing device 104 and communicative with the computing device 104, by way of one or more networks, such as without limitation the internet. Alternatively, or additionally, user interface 204 may communicate with computing device 104 using telephonic devices and networks, such as without limitation fax machines, short message service (SMS), or multimedia message service (MMS). Commonly, user interface 204 communicates with computing device 104 using text-based communication, for example without limitation using a character encoding protocol, such as American Standard for Information Interchange (ASCII). Typically, a user interface 204 conversationally interfaces a chatbot, by way of at least a submission 208, from the user interface 204 to the chatbot, and a response 212, from the chatbot to the user interface 204. In many cases, one or both of submission 208 and response 212 are text-based communication. Alternatively, or additionally, in some cases, one or both of submission 208 and response 212 are audio-based communication.

Continuing in reference to FIG. 2, a submission 208 once received by computing device 104 operating a chatbot, may be processed by a processor 220. In some embodiments, processor 220 processes a submission 208 using one or more of keyword recognition, pattern matching, and natural language processing. In some embodiments, processor employs real-time learning with evolutionary algorithms. In some cases, processor 108 may retrieve a pre-prepared response from at least a storage component 216, based upon submission 208. Alternatively, or additionally, in some embodiments, processor 108 communicates a response 212 without first receiving a submission 208, thereby initiating conversation. In some cases, processor 108 communicates an inquiry to user interface 204; and processor 108 is configured to process an answer to the inquiry in a following submission 208 from the user interface 204. In some cases, an answer to an inquiry present within a submission 208 from a user device 204 may be used by computing device 104 as an input to another function. As a nonlimiting example, memory 116 may contains instructions configuring processor 108 to perform a second scan after prompting the user for the second scan based on the guidance datum 128 generated.

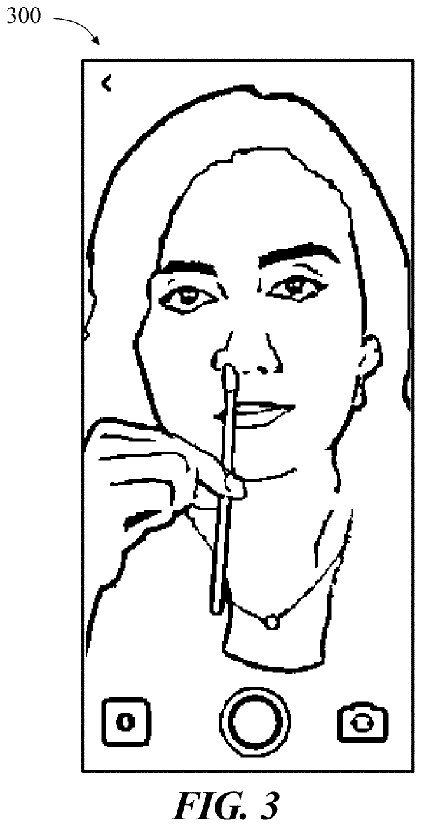

Now referring to FIG. 3, an illustrative embodiment of a scan of the user is presented. In a nonlimiting example, user may be scanned using a handheld device with an integrated optical device 112. In another nonlimiting example, user may be scanned using an external optical device 112, such as a camera connected to a desktop computer. In an embodiment, the user scan may be limited to an are surround the user's head. In other embodiments, scan may include other areas of the body. In embodiments, scan of the user may include a scan of the user's whole body.

Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example one or more head movement, arm movement, hand movement or object motion may be used as input and a guidance motion pattern may be used as output.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data to disease testing context. In some cases, user movement patterns may change depending on the type of disease being tested. In one example, without limitations, a test for COVID-19 virus may require the insertion of a swab into each nostril at approximately 2.5 cm depth and five rotations of the swab at that depth. In another nonlimiting example, a test for group a streptococcus bacteria requires the swab be inserted inside the

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively, or additionally, and with continued reference to FIG. 4, machine-learning processes as described in this disclosure may be used to generate machine-learning models 424. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include user movements such as head movements, hand movements, arm movements and object motions as described above as inputs, guidance motion patterns as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naive Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

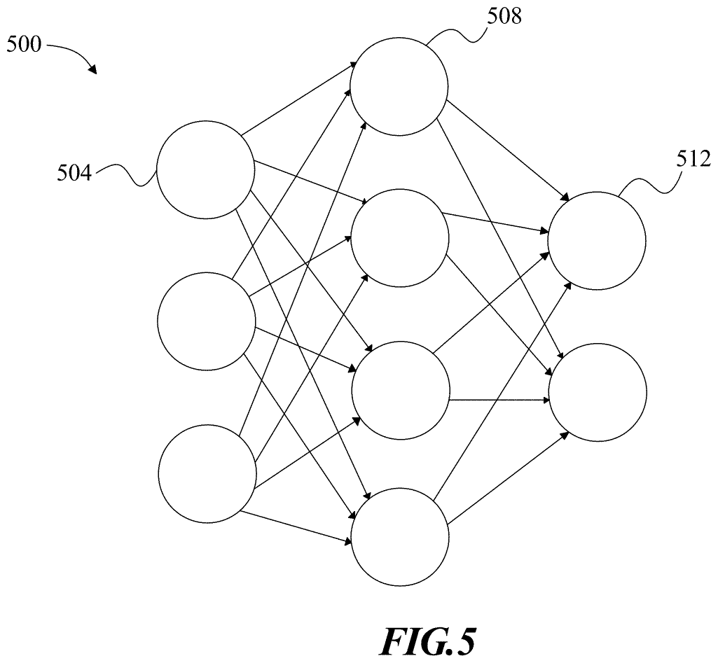

Referring now to FIG. 5, an exemplary embodiment of neural network 500 is illustrated. A neural network 500 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 504, one or more intermediate layers 508, and an output layer of nodes 512. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

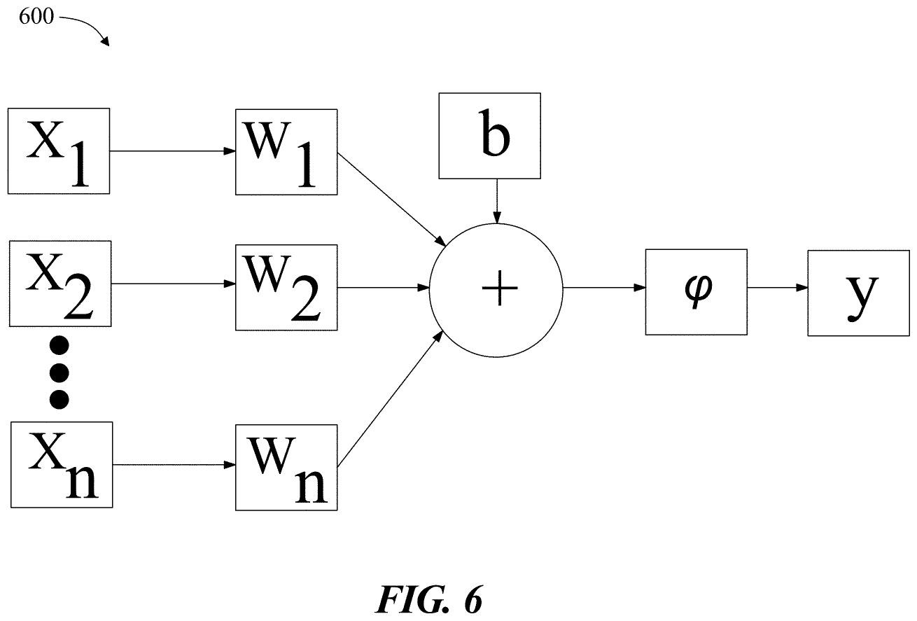

Referring now to FIG. 6, an exemplary embodiment of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function ϕ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

Referring to FIG. 7, an exemplary embodiment of fuzzy set comparison 700 is illustrated. A first fuzzy set 704 may be represented, without limitation, according to a first membership function 708 representing a probability that an input falling on a first range of values 712 is a member of the first fuzzy set 704, where the first membership function 708 has values on a range of probabilities such as without limitation the interval [0,1], and an area beneath the first membership function 708 may represent a set of values within first fuzzy set 704. Although first range of values 712 is illustrated for clarity in this exemplary depiction as a range on a single number line or axis, first range of values 712 may be defined on two or more dimensions, representing, for instance, a Cartesian product between a plurality of ranges, curves, axes, spaces, dimensions, or the like. First membership function 708 may include any suitable function mapping first range 712 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 7, first fuzzy set 704 may represent any value or combination of values as described above, including output from one or more algorithms, one or more machine-learning models, one or more sensors and a predetermined class, such as without limitation a user movement (e.g., hand movement, arm movement, object motion, and the like). A second fuzzy set 716, which may represent any value which may be represented by first fuzzy set 704, may be defined by a second membership function 720 on a second range 724; second range 724 may be identical and/or overlap with first range 712 and/or may be combined with first range via Cartesian product or the like to generate a mapping permitting evaluation overlap of first fuzzy set 704 and second fuzzy set 716. Where first fuzzy set 704 and second fuzzy set 716 have a region 728 that overlaps, first membership function 708 and second membership function 720 may intersect at a point 732 representing a probability, as defined on probability interval, of a match between first fuzzy set 704 and second fuzzy set 716. Alternatively, or additionally, a single value of first and/or second fuzzy set may be located at a locus 736 on first range 712 and/or second range 724, where a probability of membership may be taken by evaluation of first membership function 708 and/or second membership function 720 at that range point. A probability at 728 and/or 732 may be compared to a threshold 740 to determine whether a positive match is indicated. Threshold 740 may, in a non-limiting example, represent a degree of match between first fuzzy set 704 and second fuzzy set 716, and/or single values therein with each other or with either set, which is sufficient for purposes of the matching process; for instance, threshold may indicate a sufficient degree of overlap between an output from one or more machine-learning models and a predetermined class, such as without limitation a user movement, for combination to occur as described above. Alternatively, or additionally, each threshold may be tuned by a machine-learning and/or statistical process, for instance and without limitation as described in further detail below.

Further referring to FIG. 7, in an embodiment, a degree of match between fuzzy sets may be used to classify one or more user movements with a guidance motion pattern. For instance, if a user movement has a fuzzy set matching a guidance motion pattern fuzzy set by having a degree of overlap exceeding a threshold, computing device 104 may classify the user movement as belonging to the guidance motion pattern. Where multiple fuzzy matches are performed, degrees of match for each respective fuzzy set may be computed and aggregated through, for instance, addition, averaging, or the like, to determine an overall degree of match.

Still referring to FIG. 7, in an embodiment, one or more user movements may be compared to multiple guidance motion patterns fuzzy sets. For instance, user movements may be represented by a fuzzy set that is compared to each of the multiple user state fuzzy sets; and a degree of overlap exceeding a threshold between the user movements fuzzy set and any of the multiple guidance motion pattern fuzzy sets may cause computing device 104 to classify the user movements as belonging to a guidance motion pattern. For instance, in one embodiment there may be two guidance motion pattern fuzzy sets, representing respectively an angled leftward motion and an angled rightward motion. Angled leftward motion may have an angled leftward motion fuzzy set; angled rightward motion may have an angled rightward motion fuzzy set; and user movement may have a user movement fuzzy set. Computing device 104, for example, may compare a user movement fuzzy set with each of angled leftward motion fuzzy set and angled rightward motion state fuzzy set, as described above, and classify a guidance motion pattern to either, both, or neither of angled leftward motion or angled rightward motion. As used herein, “angled leftward motion” and “angled rightward motion” are exemplary descriptions of a combination of user movements guiding a swab into the nostrils from the left or right position at an angle. Machine-learning methods as described throughout may, in a non-limiting example, generate coefficients used in fuzzy set equations as described above, such as without limitation x, c, and σ of a Gaussian set as described above, as outputs of machine-learning methods. Likewise, one or more user movements may be used indirectly to determine a fuzzy set, as user movements fuzzy set may be derived from outputs of one or more machine-learning models and/or algorithms that take the aforementioned patterns and/or parameters as inputs.

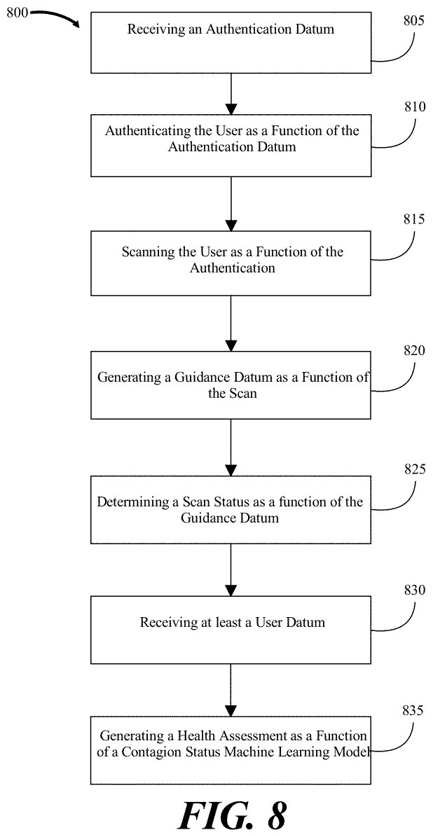

Referring now to FIG. 8, an exemplary method 800 of generating a contagion prevention health assessment is illustrated by way of a flowchart. At step 805, method 800 includes receiving an authentication datum from a user. Receiving the authentication datum may include receiving the authentication datum through a user input. As a non-limiting example, authentication datum may be receiving through a user login prompt. Receiving the authentication datum may include a 2-step authentication, consistent with embodiments described in this disclosure. Authentication datum may include an authentication token. As a nonlimiting example, authentication datum may be a user's login information. In another example, authentication datum may be generated using a two step authentication system such as using an RSA key, which is described further above.

Continuing to refer to FIG. 8, at step 810, method 800 includes authenticating the user as a function of the authentication datum. Authenticating the user may include utilizing a speech recognition model. Authenticating the user may include scanning a QR code. Authenticating the user may further include generating an authentication token as function of the QR code. In a nonlimiting example, processor 108 may scan a QR code from a test kit and generate a unique authentication token that combines the QR code and a user's login information.

With continued reference to FIG. 8, at step 815, method 800 includes scanning the user as a function of the authentication datum. Scanning the user further includes using motion recognition machine learning model 124. Motion recognition machine learning model 124 may include any machine learning model, or algorithm, disclosed herein. As a nonlimiting example, processor 108 may start scanning the user after authentication is complete and, using motion recognition machine learning 124, analyze whether user's motion matches a set of motions constituting a proper testing.

Still referring to FIG. 8, at step 820, method 800 includes generating a guidance datum as a function of the scan. Generating guidance datum may include comparing user movement patterns to guidance motion patterns as a function of the motion recognition machine learning model. In a nonlimiting example, processor 108 may analyze the users movements with a swab and , using motion recognition machine learning model 124, compare those motions to a set of motion patterns that constitute proper testing, and if the movement of the user meet a threshold, a guidance datum with a positive result is generated.

Still referring to FIG. 8, at step 825, method 800 includes determining a scan status as a function of the guidance datum. In a nonlimiting example, scan status may be a “correct” status when guidance datum 128 is within a set threshold. In another nonlimiting example, scan status may be a “failed status” if guidance datum is below a set threshold.

Continuing to refer to FIG. 8, at step 830, method 800 includes receiving at least a user datum. User datum 132 may include, but is not limited to, demographic data, travel data, exposure data, contact data, household data, vaccination status data, and the like. Receiving user datum may include receiving through a user input. Receiving the user datum may include receiving from a database. Receiving the user datum may include extracting from an immutable sequential listing. User datum may include a test result datum. As nonlimiting example, user datum 132 includes information about a user that is accessed from a database and a test result for the user, where that test result may be an input by the user, such as a checkbox.

With continued reference to FIG. 8, at step 835, method 800 includes generating a health assessment as a function of a contagion status machine learning model, where the contagion status machine learning model is configured to receive the guidance datum and the at least a user datum as input and output the health assessment. Generating the health assessment may further include using a chatbot system, described above. Generating the health assessment may further include prompting the user, using the chatbot system, for a second scan as a function of the guidance datum, performing the second scan of the user as a function of the prompt, and generating a second guidance datum as a function of the second scan. Generating the health assessment may further include generating the health assessment as a function of the second guidance datum. Generating the health assessment may further include scheduling a test appointment for the user. In a nonlimiting example, the user is prompted, by a chatbot, to input the result of the test that was performed, and if the test is negative, but the guidance datum states that testing was improperly performed, the chatbot may prompt the user to perform a second scan, where the result form that second scan, and test, are used to generate the health assessment 136.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

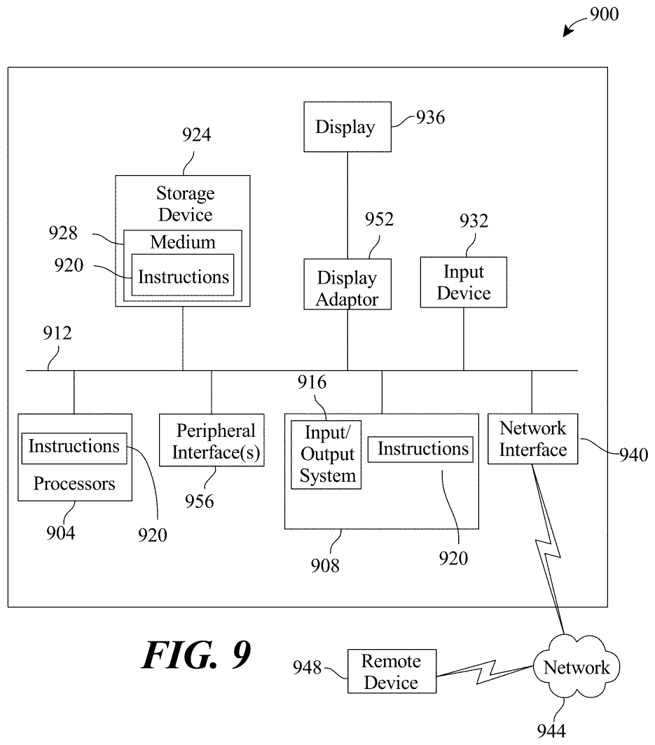

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, apparatus, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for generating a contagion prevention health assessment, the apparatus comprising:
an optical device;
at least a processor communicatively connected to the optical device; and
a memory communicatively connected to the at least a processor, the memory containing instructions configuring the at least a processor to:
receive an authentication datum from a user;
authenticate the user as a function of the authentication datum;
scan the user as a function of the authentication and using the optical device, wherein scanning the user further comprises using a motion recognition machine learning model;
generate a guidance datum as function of the scan;
determine a scan status as a function of the guidance datum;
receive at least a user datum; and
generate a health assessment as a function of the scan status and a contagion status machine learning model, wherein the contagion status machine learning model is configured to receive the guidance datum and the at least a user datum as input and output the health assessment.

2. The apparatus of claim 1, wherein the memory contains instructions further configuring the processor to generate the health assessment using a chatbot.

3. The apparatus of claim 2, wherein the memory contains instructions further configuring the processor to:
prompt the user, using the chatbot, for a second scan as a function of the guidance datum;
perform the second scan of the user as a function of the prompt, wherein scanning the user further comprises using the facial recognition machine learning model; and
generate a second guidance datum as a function of the second scan.

4. The apparatus of claim 3, wherein the memory contains instructions further configuring the processor to generate the health assessment as a function of the second guidance datum.

5. The apparatus of claim 1, wherein the at least a user datum comprises a test result datum.

6. The apparatus of claim 1, wherein the memory contains instructions further configuring the processor to schedule a test appointment for the user as a function of the health assessment.

7. The apparatus of claim 1, wherein the memory contains instructions further configuring the processor to generate the authentication datum as a function of a speech recognition model.

8. The apparatus of claim 1, wherein the memory contains instructions further configuring the processor to scan a quick read (“QR”) code.

9. The apparatus of claim 8, wherein the memory contains instructions further configuring the processor to generate an authentication token as a function of the QR code.

10. The apparatus of claim 9, wherein the authentication datum includes the authentication token.

11. A method for generating a contagion prevention health assessment, the method comprising:
receiving, by a processor, an authentication datum from a user;
authenticating, by the processor, the user as a function of the authentication datum;
scanning the user, by the processor communicatively connected to an optical device, as a function of the authentication, wherein scanning the user further comprises using a motion recognition machine learning model;
generating, by the processor, a guidance datum as a function of the scan;
determining, by the processor, a scan status as a function of the guidance datum;
receiving, by the processor, at least a user datum; and
generating, by the processor, a health assessment as a function of the scan status and a contagion status machine learning model, wherein the contagion status machine learning model is configured to receive the guidance datum and the at least a user datum as input and output the health assessment.

12. The method of claim 11, wherein generating the health assessment further comprises using a chatbot.

13. The method of claim 12, wherein generating the health assessment further comprises:
prompting, by the processor using the chatbot, the user for a second scan as a function of the guidance datum;
performing, by the processor communicatively connected to the optical device, the second scan of the user as a function of the prompt; and
generating, by the processor, a second guidance datum as a function of the second scan.

14. The method of claim 13, wherein generating the health assessment further comprises generating the health assessment as a function of the second guidance datum.

15. The method of claim 11, wherein the at least a user datum comprises a test result datum.

16. The method of claim 11, wherein generating the health assessment further comprises scheduling a test appointment for the user.

17. The method of claim 11, wherein authenticating the user further comprises utilizing a speech recognition model.

18. The method of claim 11, wherein authenticating the user further comprises scanning, by the processor communicatively connected to the optical device, a QR code.

19. The method of claim 18, wherein authenticating the user comprises generating an authentication token as a function of the QR code.

20. The method of claim 19, wherein the authentication datum includes the authentication token.
